(19) United States
(12) Patent Application Publication
(10) Pub. No.: US 2008/0063615 A1
(43) Pub. Date: Mar. 13, 2008

(54) Color changing skin sealant

(76) Inventors: John Gavin MacDonald, Decatur, GA (US); Kelly Arehart, Roswell, GA (US); Molly K. Smith, Atlanta, GA (US); Ilona Furman Weart, Woodstock, GA (US); Phillip A. Schorr, Atlanta, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/590,626

(22) Filed: Oct. 31, 2006

Related U.S. Application Data
(60) Provisional application No. 60/843,935, filed on Sep. 12, 2006.

(51) Int. Cl. A61K 8/22

(52) U.S. Cl. 424/62

(57) ABSTRACT

Iodine is used in about 80 percent of surgeries in the US to remove some level of microbial load on the skin prior to making an incision. Skin sealants are applied over skin preps to seal the skin and hold any remaining bacteria in place. Iodine produces a characteristic orange-brown color on skin. A skin sealant is provided that has a decolorant that reacts with the iodine found in most skin preps, rendering the skin prep colorless. A skin sealant containing ascorbic acid (vitamin C), Indigo Carmine or Indigo will react with the iodine, thus visually indicating where the skin prep and sealant have been applied and allowing an unobstructed view of the incision.

Pursuant to 35 U.S.C. § 120 and/or 35 U.S.C. 119(e), Applicants hereby claim priority from presently copending U.S. Provisional Application No. 60/843,935 filed on Sep. 12, 2006. The entirety of application Ser. No. 60/843,935 is hereby incorporated by reference.

### BACKGROUND OF THE INVENTION

Surgical site infections (SSI) occur following about 2-3 percent of surgeries in the United States with an estimated 500,000 incidents of SSI occurring annually, which can lead to significant patient morbidity and mortality. In addition to the negative impact of such infections on patient health, these potentially avoidable infections contribute significantly to the financial burden experienced by the health care system. SSIs result when an incision becomes contaminated by bacteria, and for most surgeries the primary source of these infection-causing microorganisms is the skin (an exception being surgeries in which the gastrointestinal tract is penetrated).

Various compositions are used to prepare the skin prior to surgery. Skin preparations or “preps” are used to remove some level of microbial load on the skin prior to making an incision. Skin sealant materials are used to protect patients from bacterial infections associated with surgical site incisions and insertion of intravenous needles. Skin preps are applied to the skin and allowed to dry to maximize effectiveness for reducing microorganisms. After the skin prep has dried, the sealant may be applied directly to the skin in liquid form. The sealant forms a coherent film with strong adhesion to the skin through various techniques based on the chemistry of the sealant composition.

Skin preps currently are predominantly povidone-iodine or chlorhexidine gluconate based formulations and may contain alcohol for fast drying and more effective killing of organisms. Time constraints in the operating room and the lack of an indicator that the prep has dried often result in the skin remaining wet when draping and/or surgery begin, creating the possibility of infection. The lack of an indicator can also negatively impact infection since the users cannot know with certainty where the prep and sealant have been applied.

Skin sealants now use a polymer composition that dries to form a film through evaporation of a solvent, for example. Other skin sealants contain monomeric units that polymerize in situ to from a polymeric film. Cyanoacrylate sealants containing alkyl cyanoacrylate monomer are an example of the latter type wherein the monomer polymerizes in the presence of a polar species such as water or protein molecules to form an acrylic film. The resulting film formed serves to immobilize bacterial flora found on the skin and prevents their migration into an incision made during a surgical procedure or skin puncture associated with insertion of an intravenous needle.

Skin sealants may contain additives such as plasticizing agents to improve film flexibility and conformance, viscosity modifiers to aid in application of the liquid composition, free radical and anionic scavengers to stabilize the product prior to use, biocidal agents to kill immobilized bacteria under the film, and the like.

Skin sealants have also been formulated with colorants to help the user apply the liquid composition uniformly to the skin, especially when large areas are to be covered. There are several problems, however, with existing colorants; addition of a colorant directly to the liquid skin sealant composition can negatively impact both in situ polymerization rates and the conversion reaction, in the case of cyanoacrylate compositions, or evaporation rates and the coalescence process in the case of polymer solution compositions. In addition, known colorants do not provide a visual cue to indicate curing of the composition has been completed. Lastly, after completion of the surgical procedure, the colorant in the sealant can obscure the wound site, making it difficult to detect redness associated with surgical site infections, bruising or leakage.

It is clear that there exists a need for a colorant that provides a visual cue to indicate coverage area and/or curing and that does not obscure the wound site.

### SUMMARY OF THE INVENTION

In response to the foregoing difficulties encountered by those of skill in the art, we have discovered that skin sealants including various decolorants may be used to indicate that a skin prep and sealant has been applied. The decolorant reacts with the iodine in the skin prep and renders it colorless. The decolorants may be added either directly to the skin sealant, incorporated into a sponge on the applicator through which the sealant is dispensed and applied to the skin, applied separately or applied simultaneously from a separate reservoir. The amount of decolorant in the sealant can be adjusted to provide a visual cue to the user of the application area and the extent of cure. Decolorants include ascorbic acid, Indigo Carmine and Indigo and many others.

### DETAILED DESCRIPTION OF THE INVENTION

Skin preparations or “preps” are used to remove some level of microbial load on the skin prior to making an incision. Skin preps are applied to the skin and allowed to dry to maximize effectiveness for reducing microorganisms. Skin preps currently are predominantly povidone-iodine or chlorhexidine gluconate based formulations and may contain alcohol for fast drying and more effective killing of organisms. Povidone iodine, available commercially as Betadine® is estimated to be used in 80 percent of surgeries as a skin preparation. Betadine® skin prep is an aqueous solution of 10 percent povidone iodine having 1 percent titratable iodine content. When Betadine® skin prep is applied to the skin, it imparts and orange-brown color.

Skin sealant materials are used to protect patients from bacterial infections associated with surgical site incisions and insertion of intravenous needles. Skin sealants are often applied directly over or on top of (Betadine®) skin preps. The sealant forms a coherent film with strong adhesion to the skin through various techniques based on the chemistry of the sealant composition.

It would be useful to medical personnel to know exactly where the skin sealant and prep were applied so that they could be sure that the appropriate area was covered. The inventors believe that providing a skin sealant that will change the color of the skin prep over which it is applied will provide valuable information for the medical professional.

A number of materials can discharge the color of iodine-based skin preps. These materials (decolorants) include ascorbic acid (vitamin C) and its derivatives, and organic oxidizing agents like peroxygen bleaches and organic oxides as further described below. In some embodiments, the discoloration reaction occurs in less than 10 minutes, more particularly less than 5 minutes, and is visible to the unaided eye.

Derivatives of ascorbic acid include ascorbyl 6-palmitate (C-16), ascorbyl 6-caprylate (C-8), ascorbyl 6-laurate (C-12), or, more broadly stated; derivatives where the 6 position of ascorbic acid has an R group where R=C1 to C18 alkyl, aryl or cycloalkyl, R=a halogen, nitro, cyano; R=heterocyclic or R=phosphate, sulfate, nitrate or chloride.

Suitable oxidizing agents are any that have a reduction potential higher than the one reaction of I₂ to 2I⁻. The standard reduction potential E° value at 25° C. and at a pressure of 1 atm for I₂+2e=2I⁻ is 0.54V. Thus oxidizing agents for use herein have a standard reduction potential E° of more than 0.54V. It should be noted that the reduction potential of this reaction at the pH of the cyanoacrylate skin sealant (pH2) is E°_{Red}=0.281V.

The standard reduction potential is a criterion well known in the chemical field for defining the oxidation/reduction power of a given material. It is for example illustrated in CRC handbook of chemistry and physics, 76^{th} edition, David R. Lide, PhD, CRC Press, page 8-21 to 8-33. A suitable way to measure the standard potential is by reference to SHE (Standard Hydrogen Electrode) by means of an electrochemical cell. This method is for instance illustrated in Kirk Othmer, Encyclopedia of chemical technology, 1981, vol. 15, page 3940. Unlike the tables which list standard potentials, values for oxidizing agents are experimental values dependent from the experimental conditions, electrodes and techniques used. Accordingly the reduction potential may be reported as experimental values, usually a half-way potential (E½ in polarography) or a peak potential (Ep in cyclic voltammetry). Whatever the conditions, electrodes, and techniques used, the oxidizing agents suitable for use herein have a reduction potential higher than the reduction potential of the reaction I₂ to 2I⁻. In other words, for defining the oxidizing agents herein the reaction I₂ to 2I⁻ is taken as a reference in the same test conditions.

Organic oxidizing agents include L-cysteine alkyl esters, particularly L-cysteine ethyl ester, titanium (III) citrate, glutathione and dithiothreitol. Other oxidizing agents suitable for use include oxygen bleaches like peroxygen bleaches or mixtures thereof. Such peroxygen bleaches include hydrogen peroxide, percarbonates, persulfates, alkyl hydroperoxides, peroxides, diacyl peroxides, ozonides, superoxides, oxo-ozonides, periodates, and salts and mixtures thereof.

Suitable peroxides include, for example lithium peroxide, sodium peroxide, potassium peroxide, ammonium peroxide, calcium peroxide, barium peroxide, magnesium peroxide, silver peroxide, titanium peroxide, iron peroxide, other alkali metal salts thereof or alkaline metal salts or mixtures thereof. Suitable superoxides include, for example, lithium superoxide, sodium superoxide, potassium superoxide, calcium superoxide, other alkali metal or alkaline earth salts or mixtures thereof. Suitable ozonides include, for example, lithium ozonide, sodium ozonide, potassium ozonide, ammonium ozonide, magnesium ozonide, other alkali metal or alkaline earth metal salts or mixtures thereof. Suitable perborates include, for example, sodium perborate, potassium perborate, ammonium perborate, or other alkali metal or alkaline earth metal salts or mixtures thereof. Suitable persulfates include, for example, sodium persulfate, potassium persulfate, ammonium persulfate as well as other alkali metal or alkaline earth metals or mixtures thereof. Other suitable peroxygen bleaches include diacetylperoxydicarbonate, 1,1-bis(tertbutylperoxy)-3,5,5-trimethylcyclohaxane, di(naphthyl) peroxide, tert-butyl perbenzoate, percarbonates like stearyl percarbonates, 20ethylhexyl percarbonate and sec-butyl percarbonate and corresponding perborates and persulfates.

Suitable diacyl peroxides have the formula: R₁—C(O)—O—O—(O)C—R₂, wherein R₁ and R₂ can be the same group or different and may be substituted or unsubstituted, saturated, or unsaturated, linear, branched or cyclic hydrocarbon groups having from 1 to 50 carbon atoms, preferably from 2 to 40 and more preferably from 4 to 18 carbon atoms. Examples of suitable diacyl peroxides are dilauryl peroxide, didecanoyl peroxide, benzoyl peroxide, benzoyl stearoyl peroxide, benzoyl decanoyl peroxide, benzoyl cetyl peroxide, di-t-butyl peroxide, diethyl peroxide, dicumyl peroxide, disteroyl peroxide, or mixtures thereof.

Suitable peroxyacids have the formula: R₃—CO₃H, wherein R₃ is a substituted or unsubstituted, saturated or unsaturated, linear or branched hydrocarbon group having from 1-25 carbon atoms or a cyclic group having from 3 to 32 carbon atoms and optionally at least one heteroatom or cyclic alkyl group having from 4 to 32 carbon atoms and optionally one heteroatom.

Other examples of decolorants include FD&C blue 2 (Indigo Carmine), D&C blue 6 (Indigo), potassium periodate (KIO₄), potassium percarbonate (KCO₃.1.5H₂O₂), sodium thiosulphate (Na₂S₂O₃), potassium perchlorate (KClO₄), hydrogen peroxide (3% is very slow, decolorizing in approximately 10 minutes, but 38% is rapid), urea hydrogen peroxide (percarbamide or carbamide peroxide) is used as a teeth brightening agent and also as an antiseptic oral cleanser. (CH₄N₂O.H₂O₂), benzoyl peroxide (used for acne treatments), potassium metabisulfite (K₂O₅S₂), potassium persulfate (K₂O₈S₂), sodium perborate (NaBO₃).

While not wishing to be held to a particular theory, it is believed that the mechanism of action is attributed to ascorbic acid and the other above reagents reacting as oxidizing agents that convert the colored iodine to the colorless iodide ion. Thus the decolorant should be an oxidizing agent having a reduction potential higher than the reduction potential of the reaction I₂ to 2I⁻, i.e. higher than E°_{red}=0.54V.

Ascorbic acid is an organic acid with antioxidant properties. Its appearance is white to light yellow crystals or powder. It is water soluble. The L-enantiomer of ascorbic acid is commonly known as vitamin C. The name is derived from a- and scorbuticus (Scurvy) as a shortage of this molecule may lead to scurvy. In 1937 the Nobel Prize for chemistry was awarded to Walter Haworth for his work in determining the structure of ascorbic acid (shared with Paul Karer, who received his award for work on vitamins), and the prize for Physiology or Medicine that year went to Albert Szent-Gyorgyi for his studies of the biological functions of L-ascorbic acid.

Ascorbate acts as an antioxidant by being itself available for energetically favorable oxidation. Many oxidants (typically, reactive oxygen species) such as the hydroxyl radical (formed from hydrogen peroxide), contain an unpaired electron and thus are highly reactive and damaging to humans and plants at the molecular level. This is due to their interaction with nucleic acid, proteins and lipids. Reactive oxygen species oxidize (take electrons from) ascorbate first to monodehydroascorbate and then dehydroascorbate. The reactive oxygen species are reduced to water while the oxidized forms of ascorbate are relatively stable and unreactive, and do not cause cellular damage.

Indigo Carmine has a molecular weight of 466.36 and consists of a mixture of disodium 3,3′ dioxo-2,2′-bi-indolylidene-5,5′-disulfonate, disodium 3,3′-dioxo-2,2′-bi-indolyldene-5,7′-disulfonate. Indigo carmine exists as a sodium salt as 5,5′-indigodisulfonic acid disodium salt.

The inventors realized that if a decolorant were used with skin sealant, it would discharge the iodine present in most skin preps, thus assisting the medical professional in knowing where the skin prep and sealant had been applied.

Decolorization reactions of iodine are known both in laboratory analysis of vitamin C in tablets and fruit juices and also as an effective method for decontaminating water when in the outdoors. Both involve the decolorization of the iodine by the reducing agent power of, for example, ascorbic acid (vitamin C) the reaction of iodine with ascorbic acid is:

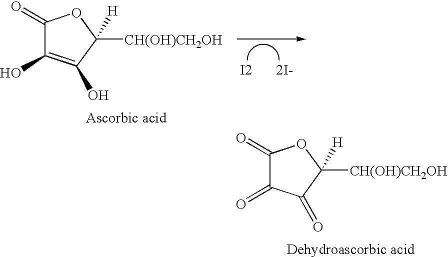

It is also possible to apply a sufficient amount of skin sealant and therefore decolorant to the iodine prep in order to achieve a visible color reduction of the Iodine to the naked eye and yet have sufficient Iodine that the residual still retains a strong antimicrobial benefit.

As noted above, there a number of ways to use a decolorant with a skin sealant: it may be mixed with the skin sealant, it may be impregnated onto a sponge or wipe which is used to apply the sealant, it may be applied separately from a separate reservoir and it may be applied simultaneously from a separate reservoir in a manner similar to the application of an epoxy.

The application of a decolorant to a carrier may be done by the “dip and squeeze” method, known to those skilled in the art. In this method, the carrier (e.g., sponge, nonwoven fabric (wipe), cotton ball or other) is placed in a bath of the decolorant and allowed to absorb the decolorant. After absorbing the decolorant, the carrier is squeezed between, for example, a pair of rollers, to force out excess decolorant.

Another method to apply decolorant to a carrier is to spray the decolorant onto the carrier. Spraying generally does not penetrate the carrier with decolorant as well as the dip and squeeze method, though it is generally faster and simpler.

Yet another method to apply a decolorant to, for example, a stack of wipes in a storage box, is to add the decolorant to the box with the wipes. U.S. Pat. Nos. 4,775,582 and 4,853,281, incorporated by reference in their entirety commonly assigned, concern a method of maintaining relatively uniform moisture in a stack of wipes. The wipes may be made from polyolefinic microfibers that have been extruded and gathered like spunbond or meltblown fibers, or a combination of both. Common materials for construction of wipers include spunbond and meltblown fibers and fabrics in various arrangements.

The term “spunbond fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns. As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface. Laminates of spunbond and meltblown fibers may be made, for example, by sequentially depositing onto a moving forming belt first a spunbond fabric layer, then a meltblown fabric layer and last another spunbond layer and then bonding the laminate in a manner described below. Alternatively, the fabric layers may be made individually, collected in rolls, and combined in a separate bonding step. Such fabrics usually have a basis weight of from about 0.1 to 12 osy (6 to 400 gsm), or more particularly from about 0.75 to about 3 osy. Multilayer laminates may also have various numbers of meltblown (abbreviated as “M”) layers or multiple spunbond (abbreviated as “S”) layers in many different configurations and may include other materials like films (abbreviated as “F”) or coform materials (see U.S. Pat. No. 4,100,324 for descriptions of exemplary “coform” materials), e.g. SMMS, SM, SFS, etc.

Applying the sealant from a separate reservoir may involve the use of dispensers developed for that purpose. One exemplary dispenser has the liquid sealant held in at least one oblong glass ampoule within a rigid nylon housing. The housing has a body and a cap that are slidably connected and it is the cap which holds the ampoule(s). In use, the two parts are moved toward each other to dispense the liquid; the cap moving into the body. Moving the parts together results in breakage of the glass ampoule(s) and dispensing of the liquid. A detent-type locking mechanism holds the body and cap together once they are moved. The locking mechanism consists of slots formed in the cap into which fits a slight protuberance or knoll of plastic formed on the inside surface of the body. Once the ampoule is broken, the liquid travels through a small piece of foam which catches any glass shards that may have been formed by the breakage of the ampoule and thence on to the tip portion of the body. The tip has a number of small holes in it to allow the liquid to pass through. The body tip has a piece of foam on the outside, held in place with a rigid plastic oval-shaped ring that snaps in place on the tip. The outer foam contacts the skin of the patient when the liquid is dispensed. Other types of dispensers may be found in U.S. Pat. Nos. 4,854,760, 4,925,327 and 5,288,159, incorporated herein by reference.

In another embodiment the skin sealant and decolorant may be applied separately to the area containing a skin prep. U.S. Pat. No. 5,928,611 describes a dispenser having a skin sealant reservoir and an active ingredient such as a cross linking accelerator or initiator disposed on a foam piece through which the sealant must pass. One could envision the use of such a dispenser having the decolorant disposed on the foam piece and the sealant passing though it as it is about to be deposited onto the skin. See also U.S. Pat. No. 6,322,852.

In yet another embodiment, U.S. Pat. No. 6,340,097 describes a dispenser having at least one crushable ampoule within the body of the dispenser which could hold more than one. This would permit one ampoule to hold skin sealant and a second to hold the decolorant. When the dispenser was used, it would break both ampoules and the sealant and decolorant would mix just before application to the skin.

In addition to being used as a traditional skin sealant, i.e. as a film forming barrier through which a surgical incision is made, the decolorant and skin sealant composition may also be used like a bandage to close and/or cover wounds, abrasions, burns, acne, blisters and other disruptions in the skin to protect them from subsequent contamination. The use of the skin sealant composition would therefore not be limited to medical personnel.

Wound protection is critical in permitting the healing process to take place. Traditional adhesive bandages and gauze wound dressings have been used by the consumer to treat/dress acute wounds or skin irritations. Such adhesive bandages are generally passive, in that they offer little or no chemical treatment for wound healing. Rather, they primarily serve to exert low levels of pressure on the wound, protect the wound from exposure to the environment, and absorb any exudates, which are produced from the wound site. Such bandages generally include a base layer, which is the layer seen by the consumer following application of the bandage to the wound. Such a layer is typically formed from a polymeric material such as a film, nonwoven web, or combination thereof, and may be perforated in some fashion to allow for flexibility and/or further breathability. This layer often includes a film component, having a top side surface which is seen by the consumer after application of the bandage to the wound site, and a bottom side surface (skin contacting surface). A skin-friendly adhesive is usually placed over the base layer bottom side surface to provide a means for attaching the bandage to the consumer. Alternatively, a separate adhesive tape is used to attach the bandage/wound dressing to the wound site, if the bandage/wound dressing is of the nonadhesive type. In the center of the base layer bottom side surface is traditionally positioned an absorbent pad for absorbing exudates from the wound. Finally, a non-stick perforated film layer is normally positioned over the absorbent pad layer, to provide a barrier between the absorbent pad and the wound itself. This allows the wound fluid to move through the perforated layer without sticking to the wound site. Typically the absorbent pad in such bandage does not include any medicinal components, although comparatively recently, bandage manufacturers have started including antibiotic agents on or within bandages to encourage wound healing.

The skin sealant composition of this invention can replace this seemingly complicated bandage construction with a single liquid treatment that will dry to a flexible coating that protects a wound much like a bandage would. Additionally, medicaments such as antibiotic agents may be blended in effective amounts with the composition to provide additional benefits in the area of microbial inhibition and the promotion of wound healing. The sealant may be applied to provide an effectively thick coating over the surface of the superficial wound, burn or abrasion. Because the to-be-treated wound is superficial and does not extend beyond the dermal layer, any polymeric residues diffusing into or forming in the wound will be naturally extruded from the skin. Generally, the sealant provides an adhesive film coating over the wound area which when set is satisfactorily flexible and adherent to the tissue without premature peeling or cracking. The coating generally has a thickness of less than about 0.5 millimeter (mm).

Sealant coatings of such thicknesses form a physical barrier layer over superficial wounds which provide protection for the wound in the same manner as a conventional bandage. Specifically, the coating provides an almost airtight, waterproof seal around the wound which does not need to be replaced when the wound gets wet. Once applied, the coating prevents bacterial and contaminant entry into the wound, thus reducing the rate of secondary infection. Generally, the adhesive coating does not limit dexterity and promotes faster wound healing. Additionally, unlike conventional bandages, the sealant naturally sloughs off the skin within 2-3 days after application and, accordingly, avoids the discomfort associated with removal of conventional bandages from the skin. However, if early removal of this polymeric coating is desired, such can be achieved by use of solvents such as acetone. Further discussion of this use may be found in U.S. Pat. No. 6,342,213.

By way of elaboration it should be noted that several wound care products are currently being marketed which contain an antiseptic benzalkonium chloride and an antibiotic mixture of polymixin B-sulfate and bacitracin-zinc. Patents in this area of technology have described the use of commonly known antiseptics and antibiotics, such as those described in U.S. Pat. Nos. 4,192,299, 4,147,775, 3,419,006, 3,328,259, and 2,510,993. U.S. Pat. No. 6,054,523, to Braun et al., describes materials that are formed from organopolysiloxanes containing groups that are capable of condensation, a condensation catalyst, an organopolysiloxane resin, a compound containing a basic nitrogen, and polyvinyl alcohol. U.S. Pat. No. 5,112,919, reported a moisture-crosslinkable polymer that was produced by blending a thermoplastic base polymer, such as polyethylene, or a copolymer of ethylene, with 1-butene, 1-hexene, 1-octene, or the like; a solid carrier polymer, such as ethylene vinylacetate copolymer (EVA), containing a silane, such as vinyltrimethoxysilane; and a free-radical generator, such as an organic peroxide; and heating the mixture. The copolymers could then be cross-linked by reaction in the presence of water and a catalyst, such as dibutyltin dilaurate, or stannous octoate. U.S. Pat. No. 4,593,071 to Keough reported moisture cross-linkable ethylene copolymers having pendant silane acryloxy groups.

A polyurethane wound coating is described by Tedeshchl et al., in EP 0992 252 A2, where a lubricious, drug-accommodating coating is described that is the product of a polyisocyanate; an amine donor, and/or a hydroxyl donor; and an isocyanatosilane adduct having terminal isocyanate groups and an alkoxy silane. A water soluble polymer, such as poly(ethylene oxide), can optionally be present. Cross-linking causes a polyurethane or a polyurea network to form, depending upon whether the isocyanate reacts with the hydroxyl donors or the amine donors. U.S. Pat. No. 6,967,261 describes the use of chitosan in wound treatment. Chitosan is a deacetylated product of chitin (C₈H₁₃NO₅)ₙ, an abundant natural glucosamine polysaccharide. In particular, chitin is found in the shells of crustaceans, such as crabs, lobsters and shrimp. The compound is also found in the exoskeletons of marine zooplankton, in the wings of certain insects, such as butterflies and ladybugs, and in the cell wall of yeasts, mushrooms and other fungi. Antimicrobial properties of chitosan have been reported against Gram positive and Gram negative bacteria, including Streptococcus spp., Staphylococcus aureus, Staphylococcus epidermidis, Staphylococcus haemolyticus, Pseudomonas, Escherichia, Proteus, Klebsiella, Serratia, Acinobacter, Enterobacter and Citrobacter spp. Chitosan has also been described in the literature to induce repair of tissue containing regularly arranged collagen bundles.

The composition may also be used to close wounds much like stitches or bandages. To be used in such a way, the composition is applied to at least one skin surface of the opposed skin sections of, for example, a suturable wound of a mammalian patient (e.g., human patient). The opposed skin sections are contacted with each either before or after application of the composition. In either case, after application of the composition, the wound area is maintained under conditions wherein the composition polymerizes to join these skin sections together. In general, a sufficient amount of the composition may be employed to cover the wound and the adjacent the skin surface of at least one of the opposed skin sections of the suturable wound. Upon contact with skin moisture and tissue protein, the composition will polymerize or, in the case of compositions utilizing partially polymerized monomers, will further polymerize, at ambient conditions (skin temperature) over about 10 seconds to 60 seconds to provide a solid polymeric film which joins the skin sections, thereby closing the wound. Generally, the composition can provide a polymeric film over the separated skin sections thereby inhibiting infection of the wound while promoting healing. Further discussion of this use may be found in U.S. Pat. No. 6,214,332.

The composition may be packaged in a “kit” form for use in medical facilities and bundled with the appropriate skin prep solution for ease of use and the convenience of the medical personnel. Kits may also include a container holding the skin sealant composition and another separate container for the decolorant as previously described. The kit may also include an applicator and means for mixing the contents of the two containers. Alternatively the decolorant may be impregnated onto a sponge which is used to apply the sealant and through which the skin sealant flows when it is dispensed. In addition, various complimentary or “mating” containers and different packaging schemes have been used for some time and are known in the art.

The following examples show the efficacy of the instant approach.

### EXAMPLE 1

0.01 g (5.68×10⁻⁵ mol) USP grade ascorbic acid (from Sigma-Aldrich Chemical Co. Inc., Milwaukee, Wis.) was dissolved in 2 g of a skin sealant (known as InteguSeal® and available from Medlogic Global, Ltd of Plymouth, England) containing n-butyl cyanoacrylate monomer (0.5% w/w), and serial dilutions of this solution were made to produce 0.25% and 0.125% solutions as well. These skin sealant solutions were then applied with a cotton swab to hydrated Vitroskin® that had previously been prepared with Betadine® skin prep (from Purdue Frederick Co., Norwalk, Conn.) Vitroskin® is available from IMS Inc., of Orange, Conn. and is hydrated over glycerol/water for 12 hours before use as described in the product instructions. Each of these three solutions caused immediate decolorization of the Betadine®skin prep-treated surface when applied in this manner, and the Betadine® skin prep was not merely transferred to the swab (i.e. the swab remained white).

### EXAMPLE 2

0.025 g (1.42×10⁻⁴ mol) ascorbic acid was dissolved in 0.8282 g of InteguSeal® skin sealant (3% w/w), and serial dilutions of this solution were made to produce 1.5%, 0.75%, 0.38%, 0.19% and 0.09% solutions. A pipette was used to apply drops of these skin sealant solutions to Betadine® skin prep-treated pig skin, and the sealant was then spread with a swab. As observed in the Vitroskin® experiment above, the Betadine® skin prep became decolorized upon contact with all of the ascorbic acid-containing solutions and no color was transferred to the swab.

### EXAMPLE 3

10 mg (2.1×10⁻⁵ mol) FD&C blue 2 (Indigo Carmine) (from Sigma-Aldrich) was dissolved in 100 ml of deionized water. 15 ml of the solution was placed in a vial and 23 mg Betadine® skin prep was added. The vial was swirled once and the color change observed. On addition of the Betadine® skin prep the mixture turned from blue to green. The green stared to fade and after 10 seconds the green color had discharged to result in a pale yellow color.

### EXAMPLE 4

22 mg (8.4×10⁻⁵ mol) of D&C blue 6 (Indigo) (from Sigma-Aldrich) was dissolved in 500 ml of deionized water with stirring for 3 hours. 15 ml of this solution was placed in a vial and 23 mg of Betadine® skin prep was added. The vial was swirled once and left to stand for observation of the color change. After 10 seconds the pale green color turned a dark green color. This final green color was not discharged by addition of ascorbic acid solution (0.5% wt/wt in water), showing that the color was not due to simple blue-yellow color mixing.

### EXAMPLE 5

Ascorbic acid was dissolved in a skin sealant (known as InteguSeal® and available from Medlogic Global, Ltd of Plymouth, England) containing n-butyl cyanoacrylate monomer to produce a 0.3 wt/wt solution. The skin sealant solution was then applied with a cotton swab to hydrated Vitroskin® that had previously been prepared with DuraPrep® skin prep. DuraPrep® skin prep is available from 3M Health Care of St. Paul, Minn. and contains iodorphor (0.7% available iodine) in isopropyl alcohol (70% wt/wt). Upon application of the skin sealant solution, the iodine color was rapidly decolored.

### EXAMPLE 6

Additional research identified other actives which were shown to decolorize iodine rapidly (all in less than 5 minutes). These compounds fall into four classes:
Derivatives of ascorbic acid
   Ascorbyl 6-palmitate and ascorbyl 6-laurate (both from Sigma-Aldrich Chemical Co. Inc. Milwaukee Wis.)
Organic oxidizing compounds
   Cysteine, and Cysteine ethyl ester (both from Sigma-Aldrich Chemical Co. Inc., Milwaukee Wis.)
Peroxygen bleaches
   Benzyol peroxide, (Sigma-Aldrich Chemical Co. Inc., Milwaukee Wis.), carbamide peroxide (GlaxoSmithKline Consumer Healthcare, Moon Township, Pa.)
Inorganic oxides
   Potassium periodate
   Potassium percarbonate
   Sodium thiosulfate
   Sodium perbromate (All from Sigma-Aldrich Chemical Co. Inc., Milwaukee Wis.)

The actives were all tested on iodine (Betadine®) and the efficacy of the decolorization visually observed and recorded. The following general testing procedure was used for all the actives.

The active was dissolved or dispersed into 1 gram of InteguSeal® skin sealant by mixing with a glass rod to yield a 0.3% wt/wt mixture. The Betadine® skinprep was swabbed onto a glass microscope slide and allowed to dry, producing a brown/yellow coating. The InteguSeal® skin sealant containing the active was then applied to a cotton swab (e.g. Q-tip®) and then applied to the Betadine® skin prep coating on the glass slide. The decolorization was observed and efficacy recorded.

As will be appreciated by those skilled in the art, changes and variations to the invention are considered to be within the ability of those skilled in the art. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

What is claimed is:

1. A decolorant for use with a skin sealant and application over an iodine-containing skin prep wherein said decolorant comprises at least an oxidizing agent having a reduction potential higher than the reduction potential of a reaction I₂ to 2I⁻.

2. The decolorant of claim 1 wherein said oxidizing agent is a peroxygen bleach, an inorganic oxide, or an organic oxidizing compound.

3. The decolorant of claim 1 wherein said decolorant is present in an amount between about 0.09 and 1.5 weight percent of the sealant.

4. The decolorant of claim 1 wherein said decolorant is mixed with said skin sealant.

5. The decolorant of claim 1 wherein said decolorant is impregnated onto a sponge which is used to apply said sealant.

6. The decolorant of claim 1 wherein said decolorant is applied separately from said skin sealant from a separate reservoir.

7. The decolorant of claim 1 wherein said decolorant is applied simultaneously with said skin sealant but from a separate reservoir.

8. The decolorant of claim 1 used with said sealant to cover wounds, abrasions, burns, acne, blisters and other disruptions in the skin to protect them from subsequent contamination.

9. The decolorant of claim 1 used with said skin sealant to close wounds.

10. The decolorant of claim 1 used with a skin sealant and packaged in a “kit” form.

11. The decolorant of claim 1 used with a skin sealant and bundled with an iodine-containing skin prep solution.

12. The decolorant of claim 1 which decolorizes an iodine-containing skin prep in less than 10 minutes.

13. A skin sealant comprising a decolorant.

14. The skin sealant of claim 12 wherein said decolorant comprises at least an oxidizing agent having a reduction potential higher than 0.54 V.

15. The skin sealant of claim 12 wherein said decolorant is ascorbic acid or its derivatives.

16. The skin sealant of claim 12 wherein said sealant is used to cover wounds, abrasions, burns, acne, blisters and other disruptions in the skin to protect them from subsequent contamination and to close wounds.

17. A medical kit comprising a skin sealant and a decolorant.

18. The medical kit of claim 17 further comprising an iodine-containing skin prep.

19. The medical kit of claim 17 wherein said decolorant is impregnated onto a sponge which is used to apply said sealant.

20. The medical kit of claim 17 wherein said decolorant is applied simultaneously with said skin sealant but from a separate reservoir.
